(19) United States
(12) Patent Application Publication
(10) Pub. No.: US 2006/0155367 A1
(43) Pub. Date: Jul. 13, 2006

(54) Micro-pleated stent assembly

(76) Inventor: Richard Allen Hines, Stilwell, KS (US)

Correspondence Address: Frank B. Flink, Jr, Esq.;Griffin, Flink, and Shelt, LLC, 8347 Fontana, Prairie Village, KS, 66207, US

(21) Appl. No.: 11/031,899

(22) Filed: Jan. 7, 2005

(51) Int. Cl. A61F 2/06

(52) U.S. Cl. 623/1.28

(57) ABSTRACT

The present invention is directed to a micro-pleated medical device assembly, preferably a micro-pleated stent assembly, comprising a tube micro-pleated to a delivery diameter suitable for intraluminal delivery. The micro-pleated stent assembly of the present invention is designed to have a substantially solid wall, and is thus particularly suited for the treatment of neurovascular aneurysms, having the ability to block the neck of an aneurysm. Sections of the micro-pleated stent may be selected for expansion to variable diameters in order to optimally fit the configuration of the vessel.

### CROSS-REFERENCE TO RELATED APPLICATIONS

Not Applicable.

### STATEMENT REGARDING FEDERALLY SPONSORED RESEARCH OR DEVELOPMENT

Not Applicable.

### COPYRIGHT NOTICE

A portion of the disclosure, including the figures, contains material which is subject to copyright protection. The copyright owner has no objection to the facsimile reproduction by anyone of the patent document or the patent disclosure, as it appears in the Patent and Trademark Office patent file or records, but otherwise reserves all rights whatsoever.

### TECHNICAL FIELD

The present invention is directed to the field of medical and veterinary stenting for endovascular treatments and, more particularly, treatment of neurovascular aneurysms.

### BACKGROUND OF THE INVENTION

A stent is a tubular medical device typically inserted into the lumen of a vessel, or other organ, to open the vessel and/or maintain the vessel in an open position to maintain flow within the vessel.

Stents can be balloon-expandable or self-expanding. A typical balloon-expandable stent is crimped around a pleated balloon to form a small diameter cylinder, and the expanding balloon expands the stent radially. Plastic deformation of the stent struts during balloon expansion results in a larger placement diameter sufficient to contact the lumen wall. Self-expanding stents, which are formed of compliant materials, are elastically compressed from their manufactured placement diameter and placed into a sleeve on the distal end of a catheter. Once the stent is in place in the vessel, the stent is pushed out of the sleeve and the stent expands radially to its original pre-compressed diameter or until it meets resistance from the artery without use of a balloon.

An aneurysm is formed when a weak spot in an artery stretches so thin that it is in danger of bursting from the pressure of the blood it contains. It forms a bulge or a ballooning area that may leak or rupture. An aneurysm that ruptures in a brain artery causes a stroke. Aneurysms that have wide openings at their base are called “wide neck” aneurisms and are the most difficult to treat. Wide neck aneurysms generally are defined as having a neck≧4 mm or a dome-to-neck ratio<2.

About 5 million people in the United States currently have a brain aneurysm, and about 25 percent of these are “wide neck” aneurysms. In the United States it is estimated that as many as 18 million people will develop a brain aneurysm during their lifetime. Every year it is estimated that more than 30,000 people suffer from ruptured brain aneurysms. Ten to 15 percent of these patients will die before reaching the hospital. More than 50 percent will die within the first 30 days after rupture. Of those who survive, approximately half suffer some permanent neurological deficit.

An aneurysm may cause pain from pressure on surrounding organs, but often aneurysms have no symptoms. Aneurysms may be discovered during routine medical exams or diagnostic procedures for other health problems, but most often people are unaware of a problem until a rupture occurs. As relatively simple, viable treatments for aneurysms are developed physicians will look for and find more silent aneurysms and treat them before they cause problems.

Neurosurgical clipping and endovascular coiling are two options physicians currently consider for the treatment of neurovascular aneurysms. The benefits of these treatments often do not outweigh the risks, especially for patients in whom remaining life expectancy is less than 20 years (those over age 60).

Neurosurgical clipping involves a craniotomy, an invasive, open surgical procedure with high risk. During this procedure, the arteries are exposed and one or more clips are applied across the neck of the aneurysm to stop blood from flowing into the aneurysm. The risk of a craniotomy is exacerbated in patients with a recent brain injury as well as in elderly or medically complicated patients. There is potential for further injury to the brain and additional neurological defect.

Endovascular coiling is a less invasive, non-surgical technique that involves inserting detachable platinum coils via a catheter into the aneurysm. The goal of endovascular coiling is to tightly pack coils inside the aneurysm to restrict blood flow within the aneurysm, and thus form a thrombus. The formation of a thrombus leaves little or no liquid in the aneurysm, eliminating the potential for the aneurysm to expand, leak or burst. The use of platinum allows the coils to be visible via X-ray. Although the endovascular coiling process plays a role in the treatment of brain aneurysms, the process has limitations. When platinum coils fill the aneurysm, the aneurysm size will remain basically the same and, therefore, it will continue to interfere with surrounding tissue. The procedure requires a long learning process due to its technical difficulty. The process is effective in only a small percentage of aneurysms, such as the small neck aneurysms where the coils are more likely to stay in place. In other aneurysms, the coils are likely to protrude into the parent vessel with risk of clot formation and embolism.

Physicians have begun using stents or balloon-stent combinations in combination with coiling to improve the effectiveness of coiling. A balloon may sometimes be used to push the coils into or pack them into the aneurysm. With stent-assisted coiling, a stent is used to line the artery and form a screen to hold the platinum coils inside the aneurysm.

For direct treatment of neurovascular aneurysms, today's balloon-expandable or self-expanding stent designs are inadequate. Substantial open spaces in the walls of self-expanding stents and balloon-expandable stents do not sufficiently cover the aneurysm to block blood flow to the aneurysm. For example, in the stent-assisted coiling procedure, physicians currently use a thin self-expanding stent developed by the Boston Scientific Corporation. This product was approved for use by the FDA in 2002 for use with embolic coils for the treatment of wide neck, intracranial, saccular aneurysms arising from a parent vessel with a diameter of ≧2 mm and ≦4.5 mm that are not amenable to treatment with surgical clipping. The flexibility of this Boston Scientific stent is derived from its very open design. It is intended to keep the coils in place, but the surface has a significant amount of open space and is not intended to block blood circulation across the neck of the aneurysm.

A stent with a greater percent solid area would restrict blood circulation into the aneurysm and trigger a thrombus in the aneurysm more effectively. In that event, the liquid aneurysm would solidify, eliminating the danger of rupture or leakage. If the aneurysm is filled with the thrombus only and no coils the aneurysm sack will shrink as the thrombus is absorbed, reducing pressure on the surrounding tissue.

Stents are generally designed as cylindrical shells comprised of interconnected elements or struts. The pattern of struts on the surface of the cylinder allows a stent to be crimped to a small diameter for delivery and to expand radially from the small delivery diameter to a larger placement diameter once positioned within the lumen. The final placement diameter of an expandable stent is generally between 2.5 and 4 times the delivery diameter. As a result, the surface of the expanded stent has a significant amount of open space. At the small delivery diameter, the metal struts of the stents cover about 50 percent of the surface area of the stent. At the expanded placement diameter, the area covered by the struts is only about 12 to 20 percent of the stent wall. Current research indicates that a dense stent will reduce flow into the aneurysm. The open area of a typical stent, then, is a limitation with respect to treatment of an aneurysm.

Several additional types of stents and methods for making stents have been described previously. For example, the documents U.S. Pat. Nos. 6,080,191, 6,007,573, and 6,669,719 discuss stents using methods involving rolled flat sheets. U.S. Pat. No. 6,361,588 discusses a helical stent that expands into a relaxed helical shape when released from a catheter. U.S. Pat. No. 6,689,159 discusses a radially expandable stent with cylindrical elements and where expansion occurs when the stress of compression is removed. U.S. Pat. No. 6,723,119 discusses a stent that is longitudinally expandable before and after expansion. These stents are a self-expanding type that expand into a cylindrical shape. A bifurcated stent design is discussed in U.S. Pat. No. 6,706,062 and U.S. Pat. No. 6,770,091 (the '062 and '091 patents) in which two portions of the stent are balloon expanded with two balloon catheters or separate pressures. Each branch of the stent is expanded once with a balloon.

Additional methods for treating aneurysms have been suggested. For example, the document U.S. Pat. No. 6,569,190 discusses a method for treating aneurysms that involves filling an aneurysmal sac with a non-particulate agent or fluid that solidifies in situ. This process leaves a permanent lump cast in the volume of the aneurysm. The lump is an undesirable side effect of solidification of the aneurysm volume.

The pleated stent assembly of U.S. patent application Ser. No. 10/695,527 filed on Oct. 28, 2003 (the '527 application) describes a stent for endovascular treatments that has advantages over other methods of treating aneurysms, in that, among other things, it provides a relatively solid area for closing off the aneurismal sac. However, the device of the '527 application is not capable of being selectively expanded to different diameters at different points along the length of the stent to conform non-cylindrical geometries that frequently exist, particularly around aneurysms. Additionally, the device of the '527 application is pleated onto and with a balloon over the entire length of the stent. The wide pleats of the device of the '527 application tends to limit the stent's ability to bend. Because of these factors, the pleated assembly of the '527 application is fairly rigid with poor longitudinal flexibility and therefore has some potential for artery damage during delivery in certain situations and some aneurysms may be located more distally than the stent/balloon assembly can reach.

Thus, a number of limitations exist in the existing technology for treatment of aneurysms. The risk associated with open surgery often outweighs the potential benefits, particularly if coiling is feasible. Coiling is limited to narrow neck aneurysm and is a technically challenging procedure requiring poking a guide wire and many, often over 20 coils into the sack of a fragile aneurysm. Coils can prolapse into the parent artery causing a life-threatening thrombus to form. Stents of the '527 application are limited to portions of the anatomy that can be reached with their poor longitudinal flexibility and inability to conform to non-cylindrical arteries.

### BRIEF SUMMARY OF THE INVENTION

The micro-pleated stent of the present invention satisfies the limitations listed above and improves upon the existing technology for treating aneurysms, utilizing a unique approach to the stenting process.

The present invention is directed to a micro-pleated stent assembly and method, for medical and veterinary use, comprising a tube, which tube is first micro-pleated and then assembled onto a balloon which is ideally compliant and ideally shorter than the stent, the assembly then being crimped to a diameter suitable for intraluminal delivery. Micro-pleating a tube refers to forming from 6 to 20 pleats, preferably about 12, in a stent so that it may be collapsed sufficiently for placement in an artery or the like. In use, the assembly is inserted into a body vessel and positioned at a target location within the lumen of a vessel. The balloon may be shorter than the micro-pleated stent to improve deliverability and to allow sections of the micro-pleated stent to be expanded to different diameters to better fit the vessel at the point of use. The surface of the tube may be solid or nearly solid or more open. The pattern may be uniform from end to end or may be different at different positions along the length or different around the circumference. The stent may have end sections that are more open than the center section and expend to a larger diameter at a given pressure than the center section so as to anchor the stent in the artery proximal and distal to an aneurysm. The pattern may consist of a patch area designed to cover the aneurism neck while minimizing blockage of micro arteries. The pattern, even if uniform around the circumference, can provide a percent solid area and average pore size that is sufficient to trigger the formation of a thrombus while being open enough with large enough pores to maintain patency in most side-branch or micro arteries. Once at the target location, the compliant balloon unpleats all or a section of the length of the micro-pleated stent and then stretches that section until it is sufficiently expanded to fit the vessel and anchor the stent. The balloon is then deflated and moved to an adjacent section of the micro-pleated stent. A second expansion of the balloon expands the adjacent section of the micro-pleated stent to the desired diameter, which may be different from the diameter obtained with the first balloon/stent expansion. Multiple balloon/stent expansions may be used to expand the entire length of the micro-pleated stent in order to properly fit the vessel. The pressure in the balloon is then released to deflate the balloon, which is then removed from the stent, the vessel and the body. Micro Therapeutics, Inc. manufactures a compliant balloon part #104-4120 that can be used in this assembly. Other compliant, semi-compliant or non-compliant balloons may be used for different micro-pleated applications. Non-compliant balloons would be used to open blocked arteries to a more nearly cylindrical shape. A semi-compliant balloon would be used to open the full length of a micro-pleated stent in a more or less cylindrical artery. An elastic or very compliant balloon would be used with multiple balloon expansions to expand a micro-pleated stent to precisely fix non-cylindrical geometry that exists around some aneurysms. One advantage then between the micro-pleated stent and the bifurcated stent designs such as in the '062 and '091 patents is that, although two balloons or two separate pressures are used to expand the bifurcated stent, the stent is not designed for multiple balloon placements and selectively controlled expansion through these multiple balloon placements and expansions, as is the micro-pleated design.

The micro-pleated stent is manufactured at a diameter the same as or only slightly smaller than the artery diameter, instead of a significantly smaller diameter like other balloon expandable stents. The micro-pleated stent would be manufactured at several specific diameters, e.g., 2.5 mm, 2.75 mm, 3.0 mm, 3.25 mm, 3.5 mm. Each stent would be capable of balloon expansion beyond the next larger as manufactured diameter so that the entire range of lumen diameters for the intended use is covered. Stents would typically be designed to expand about 20 percent after unpleating. With a maximum expansion of 20 percent above the as manufactured diameter the percent solid area would only be reduced from the manufactured value by 20 percent. For example a stent with an as manufactured solid area of 70 percent, would have a 58 percent solid area if expanded the maximum of 20 percent. The pattern on the surface of the as manufactured stent may contain a patch, a solid or nearly solid area, that could be positioned at the neck of an aneurysm, and the non-patch area of the stent would be designed to be more open to minimize blockage of micro or side-branch arteries.

The as manufactured stent is then pleated to reduce its diameter and then crimped to the delivery balloon, further reducing its diameter to the delivery-crimped diameter. When unpleated and expanded to the artery diameter it will still have a 40 to 90 percent solid surface over some or all of the surface of the stent. The percent solid area will be above the minimum needed to trigger the formation of a thrombus in the aneurysm. After unpleating, further expansion through use of the compliant balloon decreases the solid area but not by a percentage great enough to reduce effectiveness. The percent solid area of the stent surface spanning the aneurysm may be further increased by manufacturing the stent at a length longer than intended for the end-use length and then compressing the stent length to “squeeze” out most of the longitudinal space between the struts before forming the micro-pleats. Similarly the as-manufactured diameter of the stent may be crimped to a smaller diameter cylinder to squeeze out most of the circumferential space before pleating. Squeezing can produce a nearly solid surface before pleating or may be continued to force struts to overlap for even higher percent solid in the placed stent.

In one embodiment, the interconnected solid areas of the tube are substantially solid from end to end or substantially solid over a patch area only, in contrast to the stent having two anchor sections, with a middle body section, as seen with the pleated stent assembly described in the '527 application. The pattern of the micro-pleated stent is designed to allow radial expansion of the tube beyond the original diameter of the tube in some or all sections of the tube. Thus, after the tube is unpleated within the vessel, the balloon pressure can be selectively increased to expand specific sections of the tube to set it against the artery walls, covering the neck of the aneurysm, and to anchor the tube in place within the vessel. In such embodiment, the pattern in the interconnected solid areas preferably covers between 40 and 90 percent of the artery wall area covered by the stent.

The stent of the present invention is well suited to affect a cure of neurovascular aneurysms, to a greater extent than the mechanism described in the '527 application, because it gives the physician more control over the stent expansion, allowing for a more optimal fit within the artery and over the aneurysm. For example, if an artery is 3 mm in diameter on one end of the aneurysm and 4 mm in diameter on the other end of the aneurysm, the ability to expand the stent to fit 3 mm on one end of the stent and 4 mm on the other end of the stent provides a tailored fit within the artery and thus the ability to effectively treat a greater percentage of aneurysms. The micro-pleated stent assembly of the present invention can be used to treat most aneurysms, including berry, or saccular, aneurysms and fusiform aneurysms located in the neurovascular arteries, in the abdominal aortic artery and other arteries.

The stent of the present invention has more longitudinal flexibility than the pleated stents described in the '527 application because the diameter of the crimped micro-pleats is smaller and because the balloon may be shorter and more compliant. A short, more compliant, balloon may be used because it is not necessary to expand the entire stent with one balloon expansion. Additional flexibility will ensue because the stent is not pleated onto and with the balloon, decoupling the two, allowing more relative movement to improve flexibility.

The micro-pleated stent assembly of the present invention may be used in conventional stenting applications, as well as in applications wherein stenting has not been successful due to limitation of current stents and stent delivery systems. The primary application of this stent would be for a neurovascular aneurysm; however, it can be used in place of the current standard coronary stents with the additional advantage of providing a more solid area.

Using a number of different metals and alloys, the micro-pleated stent can also be designed with variable levels of radiopacity. Increasing the radiopacity will make a stent more viewable to X-rays. Less radiopacity will allow a better X-ray view of tissue growth within the stent. By using various combinations of metals and alloys, the micro-pleated stents can be designed with different levels of radiopacity, based on the desired radiopacity for a particular purpose. Neurovascular stents typically need highly radiopaque material because they are thin and because of the bone mass they must be imaged through. Coronary stents may not require a highly radiopaque material because they are typically thicker and there is less bone mass to deal with.

In one aspect, the invention is a stent comprising a tube having an original diameter and length, wherein said tube is micro-pleated along at least 6 longitudinal pleating lines to form a substantially cylindrical micro-pleated stent having a second diameter, and wherein said second diameter of said stent is less than said original diameter.

In another aspect, the present invention is a method for delivering a micro-pleated stent assembly comprising the steps of: obtaining a stent which is micro-pleated along at least 6 longitudinal pleating lines and having an internal compliant balloon; placing said stent longitudinally over a compliant balloon, thus forming a stent assembly; inserting said stent assembly into a vessel of a subject; advancing said stent assembly to a desired position within the vessel; increasing the pressure within the said balloon to unfold a portion of the length of the stent and continue to inflate the balloon until the stent section is properly seated into the artery wall; deflating the balloon and repositioning the balloon relative to the stent and repeating the stent expansion process; repeating said deflating, repositioning and inflating until the entire stent is properly expanded; and removing the balloon from the stent, the vessel and the body.

In a further aspect, the present invention is a method for forming a pleated stent comprising the steps of: forming a substantially cylindrical tube having a first diameter and a first length; placing said tube over a mandrel, said mandrel having a plurality of longitudinal ridges; forming pleats by application of blades to said tube in-between each said ridge, resulting in a stent which is pleated with a second diameter, said second diameter smaller than said first diameter. This method for forming a pleated stent may also comprise the steps of placing said stent which is pleated over a balloon compressing said stent onto said balloon, resulting in a stent with a third diameter, said third diameter being smaller than said second diameter

In summary, the micro-pleated stent assembly of the present invention can be used to treat neurovascular aneurysms by providing the required combination of (1) flexibility for delivery, (2) a sufficiently high percent solid area to cover the aneurysm and exclude blood circulation in the aneurysm, (3) relatively large openings to maintain blood flow to micro or side-branch arteries, (4) the ability to properly size the placed stent to fix its location without damage to the artery, (5) the ability for controlled, selective expansion of different sections of the stent, and (6) the ability for controlled radiopacity, a combination not found in currently available stents. In addition, a thinner, more flexible stent can be provided having more coverage with the same strength. The stent can be designed with a percent of solid coverage that varies with the length and or varies around the circumference. For example, an end with a lower percent solid area could be used to anchor the stent while a center section with a higher percent solid area could cover the aneurysm. A second example would be a pattern with a patch to be centered over the neck of the aneurysm. The high radiopacity capability of the material used for the stent and the dense pattern would allow the patch to be seen on an angiogram and rotated and positioned over the neck of the aneurysm.

### DEFINITIONS

In order to efficiently convey the meaning attributed to certain terms used in this application, the following definitions are adopted:

“As-manufactured cylindrical diameter and length”: the cylindrical diameter and length of the stent at its manufactured size before the stent is compressed and pleated.

“Crimped diameter”: the diameter of a circle that the stent will pass through after it has been crimped to the balloon.

“Compliant balloon”: used with micro-pleated stent, a compliant balloon capable of a relatively large amount of expansion beyond the original size of the balloon, by elastic deformation.

“Diameter”: when used in reference to a pleated stent or mandrel is the minimum diameter of a circle though which the item in question would pass.

“Major Diameter”: is used in a sense similar to its use with threads to refer to the outside circle around the largest part of the ridges of an object with threads or ridges.

“Minor Diameter”: is used in a sense similar to its use with threads to refer to the inside circle around the smallest part of the ridges of an object with threads or ridges.

“Non-compliant balloon”: used as coronary balloon, non-stretchy balloon that is folded into pleats and capable of expansion due to pressure causing pleats to unfold. After unfolding only small changes in diameter are produced by relatively large changes in pressure (typically 1 to 2 percent diameter increase per atmosphere of pressure up to 10 to 18 atmospheres).

“Patch”: a high percent solid area covering an area on the surface of the stent intended to be positioned at the neck of an aneurysm.

“Percent solid area”: the amount of surface area on the wall of the stent that is solid compared to the artery area covered by that area of the stent.

“Pleated stent”: a stent having 3 pleats, described in U.S. patent application Ser. No. 10/695,527 filed on Oct. 28, 2003 (the '527 application), incorporated herein by reference.

“Pleated diameter”: the diameter of a circle that the stent will pass through after it has been pleated.

“Pleated minor diameter”: outside diameter of a rod or tube that will pass through the stent after it has been pleated.

“Radiopaque”: a property of matter that allows it to be viewed with X-rays by reducing the intensity of an X-ray beam passing through it. Increasing the fluorescent radiopacity makes the stent more viewable via X-ray as a result of casting a darker shadow (bright area in the negative X-ray image).

“Squeezed cylindrical diameter and length”: the diameter and length of the cylindrical stent after it has been compressed to a smaller size from its as-manufactured size and before it has been pleated.

“Semi-compliant balloon”: used with pleated stent, a semi-stretchy balloon that is folded into pleats and capable of expansion due to pressure causing pleats to unfold, also capable of some expansion beyond the original size of the balloon with modest pressure.

### BRIEF DESCRIPTION OF THE DRAWINGS

FIG. 1 shows a longitudinal cross-section through deployed stent 10a at the site of an aneurysm 20, in an artery 30.

FIG. 2 shows a side view and an end view of an as-manufactured stent 10b and compressed stent 10c.

FIG. 3 shows a transverse cross-section of a micro-pleated stent as it is transformed from the as-manufactured 10b or compressed 10c diameter to its micro-pleated shape 10d, and to its shape 10e crimped onto a balloon 40.

FIGS. 4A through 4E show representative 2-dimensional surface patterns of as-manufactured stents.

FIG. 5 shows a longitudinal cross-section of stent 10 and balloon 40 before, at intermediate steps, and after 3 balloon expansions.

FIG. 6A shows an embodiment of a micro-pleating fixture used to form 12 micro-pleats in a stent and illustrates the mandrel 60, blades 62, movable spokes 64, and base 66.

FIG. 6B shows a close-up view of the central pleating mandrel 60, one of the radially moving pleating blades 62 and a stent 10 with one pleat 70 formed.

FIG. 7 shows an as-manufactured stent with a pattern 10 that includes a high percent solid area circular patch 12 that spans approximately half of the circumference of the stent.

### DETAILED DESCRIPTION OF PREFERRED EMBODIMENT

The present invention is directed to a micro-pleated stent, typically unpleated and expanded with an elastic balloon shorter than the stent. The unique features of the micro-pleated stent system results in the placement of a stent having a large percent solid area over the neck of an aneurysm. The stent pattern and the use of a short elastic balloon and multiple balloon expansions allow the stent to be expanded to a non-cylindrical shape to conform to the artery shape that frequently exist at the site of an aneurysm. The stent sufficiently blocks circulation into the aneurysm so as to cause a thrombus to form in the aneurysm to eliminate the danger of bursting. As the thrombus is absorbed, the aneurysm volume shrinks thus reducing pressure on surrounding tissue.

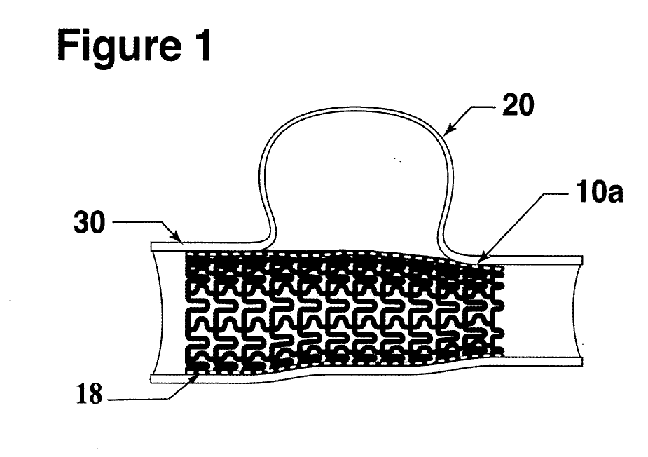

FIG. 1 shows a longitudinal cross-section of the stent 10a deployed at the site of an aneurysm 20 within the artery 30. Micro-pleated stents may be designed and used in arteries as small as about 2 mm diameter or for abdominal aortic aneurysms that may require stents as large as 30 mm diameter. For stents of the present invention used to treat neurovascular aneurysms (“neurovascular stents”), the diameter of the artery (artery diameter refers to internal diameter) is generally between about 2.0 mm and 4.0 mm. For coronary stents of the present invention, the diameter of the artery is generally between about 2.0 mm and 4.0 mm. The deployed stent will be expanded to a size slightly larger than the original artery diameter to seat or anchor the stent. Neurovascular stents will typically range in length from 8 to 14 mm. Coronary stents will typically range in length from 10 to 24 mm. Diagnostic imaging can be used to determine the appropriate diameter and length of stent 10.

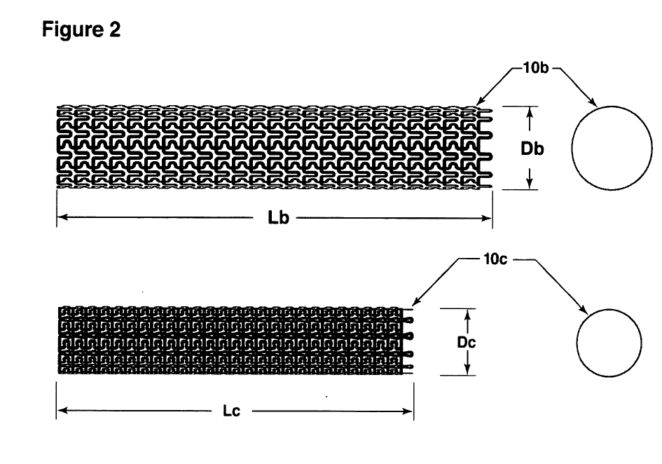

FIG. 2 shows the relative cylindrical size of the as-manufactured stent 10b, with a diameter Db and length Lb and a compressed stent 10c, with a diameter Dc and length Lc, where either Dc is less than Db, Lc is less than Lb, or both, depending upon which compression is performed. Compressing is optional. If a stent is to be compressed, it would be manufactured at a size larger than if it were not to be compressed. Thus, the size before pleating would be the same regardless whether the stent is compressed. A stent may be compressed prior to forming micro-pleats to increase the percent solid area in the deployed stent. Compressing can reduce spaces between struts below the minimum spacing that can be directly manufactured. Compressing may also be used to force struts to overlap so that the deployed stent 10a may have a larger percent solid area, near 100 percent, even after expansion beyond the compressed diameter.

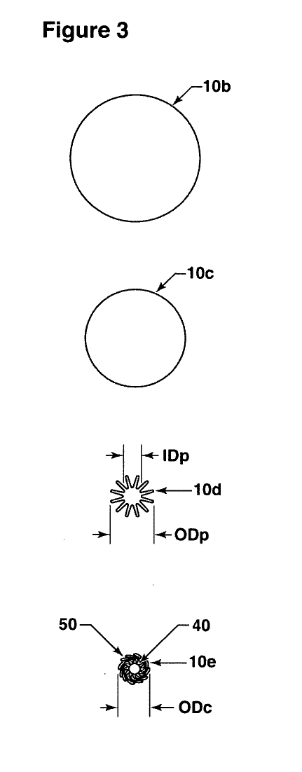

FIG. 3 shows the relative diameter and shape of the stent as it is transformed from its as manufactured diameter 10b, or its compressed diameter 10c, into its pleated shape 10d, and finally to its crimped shape 10e on balloon 40.

The stent may be laser cut from a thin-walled tube. The relatively sharp bends in the pleating process require an appropriate combination of wall 18 thinness and ductility. Since aneurysm stents cure aneurysm by simply reducing blood circulation into the aneurysm, there is no significant strength requirement for the stent, beyond that necessary to withstand a vascular spasm, allowing the stents to be very thin. A typical pure gold neurovascular aneurysm pleated stent will be between 10 and 25 microns thick. Sharp bends in thick material could cause the material to fail on pleating or unpleating. Thick material would also prevent micro-pleats from being formed. The tube material must also be ductile so that plastic deformation occurs as the stent diameter is stretched after unpleating.

Electroforming may be used to form the tube by electroplating on a cylindrical sacrificial mandrel. The pattern could be laser cut before or after mandrel removal. Other machining or etching operations could be employed to form the stent. A preferred method of forming the stent uses the electroforming processes taught in U.S. Pat. No. 6,019,784 (the '784 patent), PROCESS FOR MAKING ELECTROFORMED STENTS by Hines and the cylindrical photolithography process taught in U.S. Pat. No. 6,274,294 (the '294 patent), CYLINDRICAL PHOTOLITHOGRAPHY EXPOSURE PROCESS AND APPARATUS by Hines.

Electroforming is well suited to the fabrication of micro-pleated stents because ductile, biocompatible, radiopaque materials can be electroplated. Many commercial electroplating bath formulations are available. Gold and silver can be used for neurovascular aneurysm stents with a wall 18 thickness ranging from 10 to 60 microns, with 20 to 40 microns being preferred. Stronger deposits likes gold alloys or platinum may be thinner. Electroforming and photolithography are well suited for fabrication of thin-walled stents with fine spaces. Since the electroforming process starts with zero thickness and the thickness is controlled by the length of the run, thin stents are easy to produce. Thin stents do not require thick resist and thin resist can be more easily imaged to fine lines and spaces. Stents have been electroformed from gold, silver, nickel, copper, tin and platinum. Many other metals and alloys could be used as will be apparent to one skilled in the art of electroplating. Biocompatibility and ductility limit the choices. Heat treating may be used to improve the ductility of some electroforms.

FIG. 3 shows the stent 10b or 10c being transformed to the pleated shape 10d. FIG. 2 shows stent 10b at the as-manufactured diameter and at the optionally compressed form stent 10c. The length may be compressed by sliding the stent over a cylindrical mandrel and forcing the ends of the stent closer together to “squeeze” out longitudinal space between the struts. Similarly the diameter may be compressed in one or more steps by crimping the stent onto a cylindrical mandrel slightly smaller than the diameter of the stent. Compressing the stent prior to pleating will increase the percent solid area in the final placed stent. The optional compressing step will not be needed if the manufacturing process can directly produce a cylindrical tube with the needed flexibility for delivery and a percent solid area sufficient to trigger a thrombus in the aneurysm.

Referring to FIGS. 2 and 3, after manufacturing to form stent 10b or compressing to form stent 10c, the cylindrical stent is micro-pleated to reduce its diameter and take the form of 10d. The term micro-pleating refers to the multi-pleating process described in this application. Micro-pleating is performed independent of the balloon, which balloon is inserted separately after the pleating operation. Thus, micro-pleating is substantially different than the pleated stent of the '527 application. The pleated stent of the '527 application typically involves 3 pleats in the stent that is pleated on to and with a semi-compliant balloon. Micro-pleating consists of 6 to about 20 small pleats and the stent is not pleated with a balloon. In the preferred embodiment stent 10b is manufactured at a diameter (Db or Dc, as applicable, see FIG. 2) between 2.5 and 4.0 mm. Preferably, micro-pleated stents would be manufactured in a range of incremental diameters. The physician would select a stent 10 with a pre-pleated diameter, Db or Dc, slightly smaller than the smallest artery just proximal or distal to the aneurysm to be covered by the stent. The length, Lb or Lc, would be chosen to be long enough to span the aneurysm and allow sufficient length to anchor the stent 10 into the round artery on both sides of the aneurysm. Typical micro-pleated neurovascular aneurysm stents will be between 6 and 14 mm long, Lb or Lc, as applicable. A 10 mm long stent will work for most aneurysms. The shorter the stent the more deliverable it will be. Shorter stents will also reduce potential problems with blocking side-branches. More open anchor sections at the ends of the stent will also reduce side-branch problems.

Referring to FIG. 3, 6 to twenty pleats are formed in stent 10b or 10c forming micro-pleated stent 10d. Twelve pleats are used in the preferred embodiment. A large number of pleats tends to result in uniform expansion as the expanding balloon pushes initially only on the internal vertex of each pleat and then contacts the stent surface between the pleats only as the diameter approaches the unpleated diameter. Enough pleats are needed so that the central opening in the pleated stent is large enough to accept the balloon and not too large to prevent uniform deformation during crimping. Fewer pleats will also result in sliding of the balloon against the interior surface of the stent with the potential for non-uniform expansion. Mechanical constraints to the bending process based on the diameter and wall thickness of the stent tend to limit the maximum number of pleats.

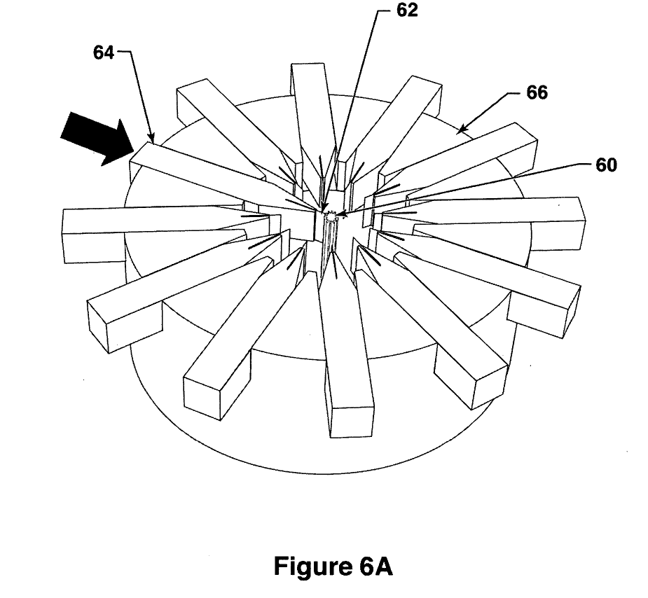

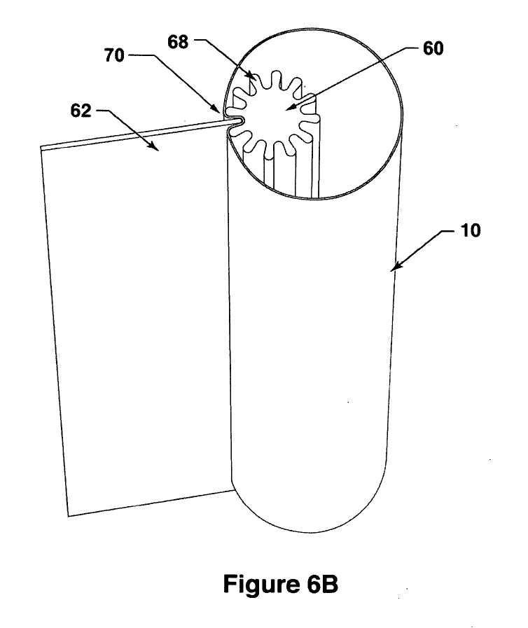

Micro-pleating may be accomplished as shown in FIGS. 6A and 6B, by threading the stent over a 12-tooth gear-shaped cylindrical mandrel 60. For example, for a 3 mm diameter stent, a mandrel 60 with a major diameter of 1.5 mm preferably is used. The gear shape may be manufactured into the mandrel by any micromachining means, such as by wire electron discharge machining (EDM). Preferably the perimeter of the gear-shaped mandrel essentially matches the circumference of the stent just prior to pleating, so that the circumferential length stent material substantially matches the surface of the mandrel after pleating. Longitudinal blades 62 are used to bend the stent 10 into the mold formed by the gear. Pleating reduces the major diameter of the stent to essentially the major diameter of the mandrel. The minor diameter interior to the stent must be large enough for the balloon to fit inside.

FIG. 6 shows a fixture used to form twelve pleats in a stent. Twelve spokes 64 are constrained to move radially by a base 66. FIG. 6B shows the center of the pleating fixture at higher magnification. The twelve toothed mandrel 60 is located in the center of the fixture. The stent 10 to be pleated would be located over the mandrel 60. Each finger terminates with a thin blade 62, typically about 0.001 inch thick, that is parallel to the axis of the mandrel and arranged so that the blades 62 will nest into the slot between the gear-shaped mandrel teeth 68. As the blades move toward the center they force the stent to bend into the teeth 68 of the mandrel 60. The circumference of the gear shaped mandrel equals the circumference of the stent and the major diameter of the mandrel equals the major diameter of the pleated stent minus two times the wall thickness. The cross section of the gear 60 determines the cross-section of the stent before crimping on to a balloon 40.

To pleat a stent using the fixture the stent 10 is threaded over the mandrel 60. One blade 62 is moved in order to mold one pleat 70 into the stent as shown in FIGS. 6A&B. The first blade 62 is held in place while the opposing blade 62, 180 degrees away, is moved into contact the stent and then moved in further to form and hold the second pleat 180 degrees away from the first pleat. The process preferably is observed through a microscope so that minor adjustments can be made to insure that the pleats divide the stent into equal portions. The process is repeated for the two blades, 62, 90 degrees away from the first two. At this point in the pleating process the stent 10 cross section is like a four-leaf clover. The final step is to move the remaining 8 blades toward the central mandrel forcing the stent to configure to the 12-toothed gear. Different gears are used for different pre-pleating diameters. A 3 mm diameter stent is typically pleated to a 12-pointed star pattern using a gear with a major diameter of 1.5 mm. The fixtures may use interchangeable gears for different diameter stents and the fingers may carry different lengths blades appropriate for the length of the stent being pleated.

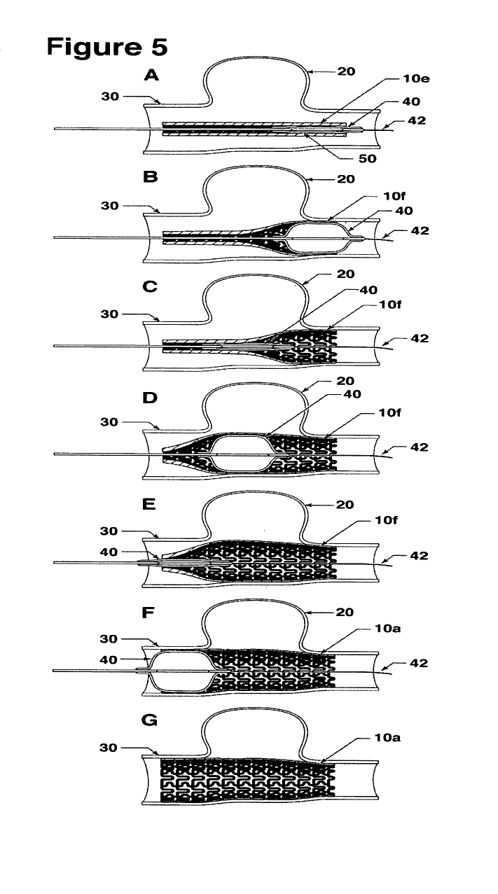

The micro-pleated stent 10d is slid off the gear and slid over a compliant balloon 40 on a balloon catheter as shown in FIGS. 3 and 5. In the preferred embodiment, the balloon will be shorter than the stent. Once the stent 10d is position over the balloon 40, the stent is crimped onto the balloon further reducing its diameter and securing it to the balloon for delivery as shown in FIG. 3 as 10e. Compliant balloons approximately 0.8 mm in diameter are available from Micro Therapeutics, Inc. (PN 104-4120). Once the micro-pleated stent is over the balloon, the major diameter of the micro-pleated stent is further reduced by crimping to the geometry shown in FIG. 3. Crimping the stent to the balloon may be accomplished using standard stent crimping tools such as the hand operated or automated crimp tools manufactured and sold by Machine Solutions Inc. The tensioned belt of the cylindrical exposures machining of the '294 patent also works well to uniformly crimp the stent to a balloon. A typical 12-pleated stent capable of expanding to 4.0 mm will be crimped to about 1.3 mm.

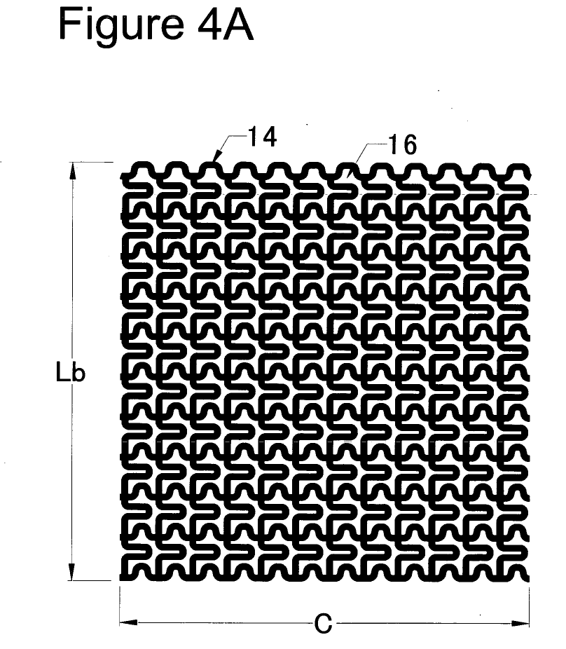

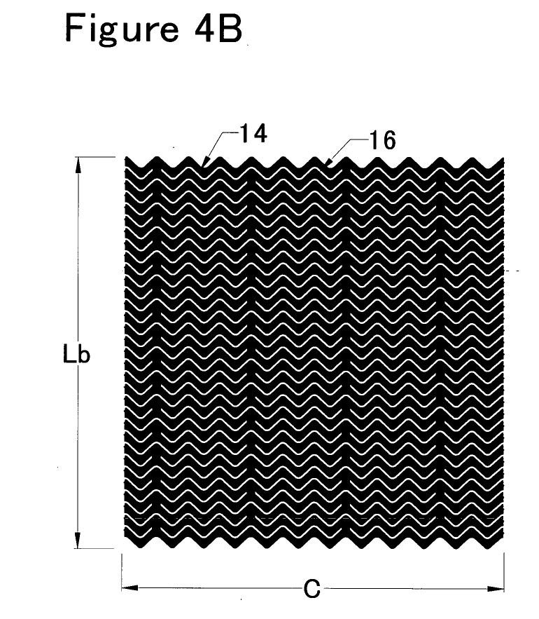

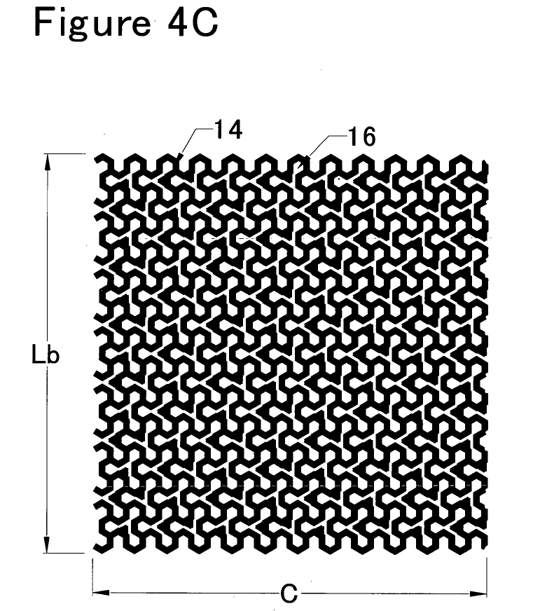

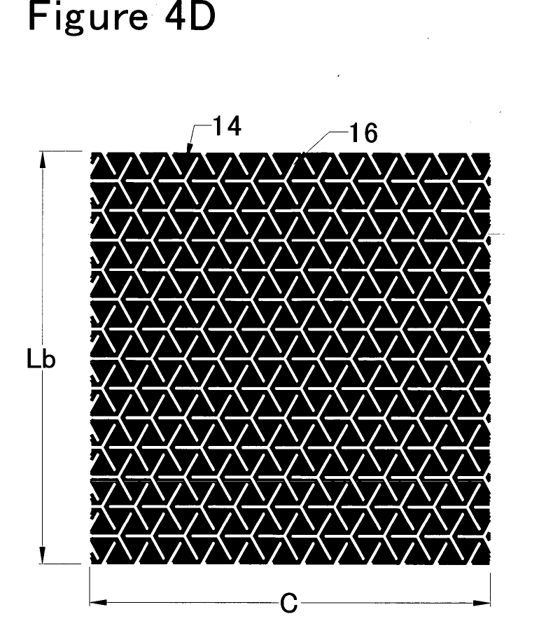

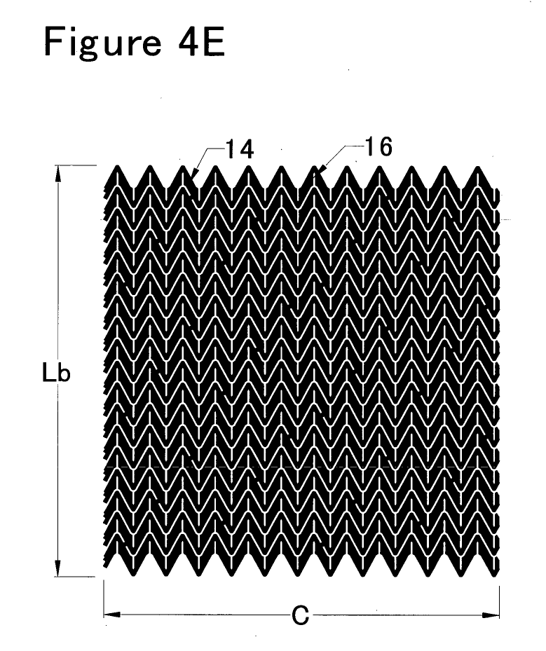

As illustrated in FIGS. 4A-E, the pattern on the surface of the stent is made up of solid areas 16 and open spaces 14. Many factors and trade-offs are considered for design. FIGS. 4A-E illustrates several of the designs that have been built and tested. The pattern must be compatible with the pleating process. Long loops that may bend away from the surface are to be avoided. The pattern must provide longitudinal flexibility for delivery so that the stent can conform to the artery. The pattern must be sufficiently dense to trigger a thrombus. Features in the pattern must be compatible with the minimum line and space capability of the manufacturing process. Electroforming can produce features down to about 25 microns. Patterns can be designed with compression in mind.

Preferred design features of stent patterns are illustrated in FIGS. 4A-E, as discussed further herein. All of the patterns shown allow for non-cylindrical expansion beyond the un-pleated size and all have patterns compatible with forming micro-pleats, preferably 12 pleats. The precise pattern utilized in a particular situation may be extrapolated to fewer or a greater number of pleats. Each of these FIGS. 4A through 4E overall show a pattern for a stent wall 18 and are oriented consistent with the stent length Lb and circumference C dimensions. The patterns further show solid areas 14 defining open spaces 16 to form the overall stent wall 18.

FIG. 4A shows a relatively open pattern that will reduce the possibility of blocking side-branch or micro arteries. The short loops in the circumferential bands provide sufficient resistance to expansion to stretch out the pleats while still providing sufficient radial expansion to properly seat the stent to the artery wall. The longitudinal loops provide longitudinal flexibility for delivery and maintain the structural integrity of the stent.

FIG. 4B illustrates a relatively dense (high percent solid) pattern with very good longitudinal flexibility. The flexibility results from only two longitudinal connectors per circumferential band. The connectors on alternate bands are located 90 degrees from connectors on adjacent bands to provide a “universal joint” type of flexibility.

FIG. 4C and FIG. 4D illustrate a moderately dense and a high density pattern based on a hexagonal close packed formation. The hexagonal pattern with tri-fold symmetry is well suited for expansion to a non-cylindrical geometry. Hexagonal based patterns can provide good longitudinal flexibility for delivery and excellent structural integrity.

FIGS. 4D and 4E illustrate how an open space 16 can be used to concentrate the stress caused by balloon expansion at selected points to minimize spring back and control the compliance of the stent with minimal impact on the percent solid area.

The pattern of a stent intended to treat a saccular aneurysm must cover the neck of the aneurysm with a pattern that has a large enough percent solid area to trigger a thrombus in the aneurysm but it is important not to block micro or side-branch arteries. Some aneurysms are found in arteries that have micro arteries, or side-branch arteries near the aneurysm that will be covered by the stent and other aneurysms are found in arteries that are free of side-branch complication. Using a stent with a length no longer than necessary to adequately cover the neck of the aneurysm will minimize the potential side-branch problem. To further reduce the potential for blocking side-branch or micro arteries the pattern can be designed with the minimum percent solid area necessary to trigger a thrombus (20 to 40% solid) while minimizing the potential to block side-branch arteries. Additionally, the average width of the open spaces can be tailored to help maintain flow in micro arteries while still trigging a thrombus in the aneurysm.

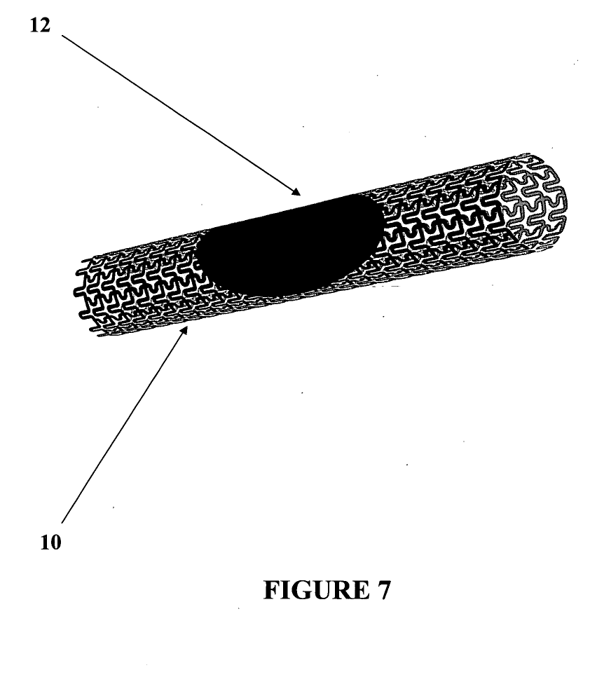

In cases with side-branch or micro arteries located directly across from the aneurysm an alternative approach to minimize unwanted blockage of side arteries is to pattern the stent with a high percent solid area patch to cover the neck of the aneurysm and to pattern the remainder of the stent with a much lower percent solid area to reduce the potential of blocking side-branch arteries. An example patch 12 is shown in FIG. 7 as a portion of an as-manufactured stent 10 with a pattern that includes a high percent solid area circular patch 12 that spans approximately half of the circumference of the stent. Stents with a patch must be delivered on a catheter that allows the stent to be rotated prior to final balloon expansion of the unpleated stent. The use of a highly radiopaque material like gold will facilitate alignment of the patch with the neck of the aneurysm. Only pleated stents that unpleat from the delivery diameter, rather than being balloon expanded, can accommodate a non-expendable area that may cover more than 50 percent of the circumference of the expanded delivered stent.

Stents with a uniform pattern over a full 360 degrees of the surface obviously do not require rotation. Full 360 degree uniform stents will be preferred for aneurysms located in arteries with minimal micro or side-branch arteries.

Patterns are designed to cover the range of balloon expansion that will be necessary after unpleating. Balloon expansion after unpleating reduces the percent solid area and in general is to be kept to a minimum, but some expansion is necessary to accommodate the variations in diameter found in the neurovascular anatomy, particularly around an aneurysm and to accommodate incremental steps in the manufactured balloon/stent assembly. Since in most cases the stent must conform to an anatomy that is not cylindrical, the stent pattern must stretch in both the circumferential direction, like standard balloon expandable stents, and must also stretch longitudinally at local points. Most stent patterns when viewed as a two-dimensional pattern, as shown in FIGS. 4A-E, are arranged on a rectangular grid. As can be seen in FIGS. 4C and 4D, the pattern may also be laid out with tri-fold symmetry based on a hexagonal grid. This type of pattern allows the surface to stretch more in all directions to fit the anatomy around an aneurysm.

FIGS. 4A through 4E are representative of a 2-dimensional surface pattern of an as-manufactured stent. As shown in FIGS. 4A-E, the surface pattern of stent wall 18 is preferably comprised of a pattern of interconnected solid areas 14 defining open spaces 16 therebetween. Because the tube of the present invention transitions between its crimped diameter and its delivery diameter primarily by pleating and unpleating, rather than by radial expansion, the wall of the tube of the present invention may be substantially solid. Ninety percent solid stents have been shown to trigger a thrombus in manufactured aneurysms in an animal. Additional tests will be necessary to determine the lower limit that will reliably trigger a thrombus. It is expected to be around 30 percent solid.

Open spaces 16 provide the longitudinal flexibility necessary for delivery and allow the circumference of the stent to be balloon expanded after unpleating until the stent is properly seated against the artery wall. The open spaces also facilitate stent 10a being covered with, and imbedded in, new body tissue and can be designed to minimize blockage of side-branch arteries or micro arteries.

FIG. 5, A through G show a longitudinal cross section before and after 3 balloon 40 expansions.

FIG. 5A shows the micro-pleated stent 10e/balloon 40 assembly 50. Note that the balloon is shorter than the stent.

FIG. 5B shows the partially-deployed micro-pleated stent 10f after the first balloon expansion. Note that the balloon expansion of the distal end of the stent also pries open the proximal end of the stent so that the balloon 40 can be repositioned. The first expansion anchors the stent into round artery distal to the aneurysm.

FIG. 5C shows the deflated balloon repositioned for the second expansion.

FIG. 5D shows the cross-section after the second balloon expansion. The expansion is shown with a larger diameter than the first expansion to show that the expansions do not need to form a cylindrical geometry.

FIG. 5E shows the deflated balloon positioned for the final expansion.

FIG. 5F shows the geometry of stent 10a after the final expansion. Again, the larger diameter in the final expansion is only intended to show that different diameters are possible with each expansion. The actual configuration will be whatever is necessary to anchor the stent and cover the aneurysm.

FIG. 5G shows the cross-section of the micro-pleated stent after the balloon 40 has been removed.

In FIG. 5A, micro-pleated stent assembly 50 is advanced to a desired position within the artery using a guide wire. The radiopacity of the stent aids in positioning the stent at the aneurysm. As in FIG. 5b, once stent assembly 50 is positioned at the desired target location within the vessel, pressure within balloon 40 is increased to expand the compliant balloon and unpleat stent to stent 10e. The expanded balloon could be nearly spherical or could be a short cylinder. Initially the internal points of the pleats will be in contact with the balloon. As the balloon expands, the “V” shaped pleats will unfold. As the pleats unfold, more of the inner surface of the stent will contact the balloon. As a section of the length of the pleated stent expands to a circular cross-section, the balloon contacts nearly 100 percent of the circumference. As the balloon is expanded beyond the as-manufactured diameter of the stent, the stent expands by deformation of the pattern that has been designed and formed into the stent. Expansion beyond the as-manufactured diameter will reduce the percent solid area of the stent. However, since expansion from the as-manufactured or compressed diameter will be small (less than 50 percent), the reduction in solid area will be small. Local plastic deformation of the pattern will maintain the expanded size with little spring back.

FIG. 5G shows the deployed stent after the balloon and guide wire have been removed.

In the preferred embodiment a pressure of about 1 to 2 atmospheres in the balloon will unpleat the section of the stent over the balloon. Balloon pressures up to 4 to 10 atmospheres will be needed to expand the typical micro-pleated stent about 20 percent beyond its pre-pleated diameter. The stent pattern combined with the wall 18 thickness and the material yield strength will determine the compliance of the stent. Referring to FIG. 5, the pressure within balloon 40 is increased based on visual angiographic feedback to provide the optimum amount of expansion, minimizing damage to the artery, while securing stent 10 in place. This will typically require expansion of the selected sections between about 0 and 20 percent beyond the pre-pleated diameter.

The micro-pleated stent of this invention may be used for coronary stent application as described in the '527 application. A micro-pleated coronary stent could be configured to look and function like today's coronary stents with a low percent solid area. Alternatively micro-pleated coronary stents could be designed to have high percent solid area and a thinner wall. A thin-wall, high surface area stent could provide strength equal to thick-wall, low percent solid stents. Thinner walls or struts will block less of the artery lumen. Coronary stents with high percent solid area would also provide more area to carry more of a drug if the stent is configured for drug elution. The small spaces and high percent solid area would deliver the drug more uniformly to the tissue. The electroforming process of U.S. patent application Ser. No. 10/452,891 (the '891 application) could be used to grow a porous layer on the as-manufactured stents of this invention. If the micro-pleated stent is electroformed, a porous drug-eluting layer could be grown in the same electroplating bath.

A stent 10b, see FIG. 2, of the present invention may be formed by any process capable of forming the desired stent pattern. In the preferred embodiment, stent 10b is formed by electroforming, as described in the '784 patent and '294 patent and the '891 application, which are hereby incorporated by reference. Stent 10b can alternatively be formed by any means known in the art or hereafter developed. For example, thin-walled cylindrical tubes may be formed on a cylinder by electroplating, vacuum evaporation or sputtering. The thin-walled tube thus formed can be patterned using cylindrical photolithography and etching of the unprotected material. The mandrel can then be dissolved to free the stent. Alternatively, the thin-walled tubes can be machined or laser machined to form the desired pattern.

Referring to FIG. 5, micro-pleated stent assembly 50 (stent and balloon) is delivered to the desired location by first inserting micro-pleated stent assembly 50 into the appropriate vessel in the body of a subject using conventional methods. The subject may be a human or other animal. It should be understood that the micro-pleated stent assembly of the present invention may be inserted and delivered into arteries, other types of vessels and other organs having a lumen. As used herein, the term “vessel” includes any vessel or other organ having a lumen, unless otherwise specified.

The micro-pleated stent assembly of the present invention can be used in a wide variety of applications. From the foregoing it will be seen that this invention is one well adapted to other applications, which are obvious and inherent to the invention. For example, it should be understood that the micro-pleated stent assembly of the present invention can be used for other applications and to treat other types of aneurysms and vascular conditions. In addition to use with stents, the micro-pleated medical device assembly can be used with other tubular medical devices. Further, when used herein, “medical device” is meant to refer to medical devices used to treat humans and veterinary devices used to treat animals.

Since many possible embodiments may be made of the invention without departing from the scope thereof, is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and sub-combinations are of utility and may be employed without reference to other features and sub-combinations. This is contemplated by and is within the scope of the claims.

What is claimed and desired to be secured by Letters Patent is as follows:

1. A stent comprising:
a tube having an original diameter and length, wherein said tube is micro-pleated along at least 6 longitudinal pleating lines to form a substantially cylindrical micro-pleated stent having a second diameter, and wherein said second diameter of said stent is less than said original diameter.

2. The stent as claimed in claim 1 where the stent is expandable to a placement diameter, in response to pressure driven expansion of an internally located compliant balloon.

3. The stent as claimed in claim 2, wherein said placement diameter is larger than said original diameter.

4. The stent as claimed in claim 2 wherein said placement diameter is essentially the same as the original diameter.

5. The stent as claimed in claim 2 wherein said balloon has a length that is shorter than the length of said tube.

6. The stent as claimed in claim 2 where said tube has a wall which is patterned for longitudinal flexibility where said pattern is comprised of a pattern of interconnected solid areas defining open spaces therebetween.

7. The stent as claimed in claim 2 where said pattern allows for radial expansion of said stent beyond said original diameter to a placement diameter in excess of said original diameter by plastic deformation of the stent.

8. The stent as claimed in claim 7 where said pattern is essentially uniform and has tri-fold symmetry forming a hexagonal grid pattern.

9. The stent as claimed in claim 7 where said pattern is a non-uniform pattern having first portions in which the open spaces defined by solid areas are sufficiently open to allow for passage of blood to branches and micro-arteries and having second portions where the open spaces defined by solid areas are sufficiently closed to allow formation of a thrombus.

10. The stent as claimed in claim 7 where said radial expansion beyond said original diameter is between 0 and 50 percent of original diameter and is in response to pressure-driven expansion of said internally located compliant balloon.

11. A stent of claim 6 where said stent is for the treatment of an aneurysm.

12. A stent of claim 6 where said stent is for the treatment of a perforated vessel.

13. A stent of claim 6 where the percent solid area on most of the surface of said stent is greater than 30 percent solid at the placement diameter.

14. A stent of claim 6 where the percent solid area on essentially all of the surface of said stent is greater than 40 percent solid and less than 90 percent solid at the original diameter.

15. A stent/balloon assembly consisting of the stent of claim 2 crimped onto said compliant balloon.

16. An assembly of claim 15 wherein said balloon is shorter than said stent.

17. The stent of claim 1, wherein said tube is formed from an electroformed metal.

18. The stent of claim 1, wherein said tube is composed of a metal.

19. The stent of claim 18, wherein said metal is gold, silver, platinum, copper, silver, tin or various combinations or alloys thereof.

20. The stent of claim 1, wherein said tube is formed from a biocompatible plastic.

21. The stent of claim 1, wherein said tube is formed from a bioabsorbable material.

22. The stent of claim 1, wherein the number of said pleating lines is between 8 and 16.

23. The stent of claim 1, wherein said stent is capable of being expanded by means of an internal balloon to different diameters at different positions along the axis of said stent.

24. A method for delivering a micro-pleated stent assembly comprising the steps of:
obtaining a stent which is micro-pleated along at least 6 longitudinal pleating lines and having an internal compliant balloon;
placing said stent longitudinally over a compliant balloon, thus forming a stent assembly;
inserting said stent assembly into a vessel of a subject;
advancing said stent assembly to a desired position within the vessel;
increasing the pressure within the said balloon to unfold a portion of the length of the stent and continue to inflate the balloon until the stent section is properly seated into the artery wall;
deflating the balloon and repositioning the balloon relative to the stent and repeating the stent expansion process;
repeating said deflating, repositioning and inflating until the entire stent is properly expanded;
removing the balloon from the stent, the vessel and the body.

25. The method of claim 24, wherein said vessel is an artery and said desired position is adjacent to an aneurysm.

26. A method for forming a pleated stent comprising the steps of:
forming a substantially cylindrical tube having a first diameter and a first length;
placing said tube over a mandrel, said mandrel having a plurality of longitudinal ridges;
forming pleats by application of blades to said tube in-between each said ridge, resulting in a stent which is pleated with a second diameter, said second diameter smaller than said first diameter.

27. A method for forming a pleated stent as claimed in claim 26 further comprising the steps of:
placing said stent which is pleated over a balloon;
compressing said stent onto said balloon, resulting in a stent with a third diameter, said third diameter being smaller than said second diameter.

28. A method for forming a pleated stent as claimed in claim 26 wherein the number of pleats is at least 6.

29. A method for forming a pleated stent as claimed in claim 28 wherein the step of:
compressing said tube to a second diameter and second length;
occurs prior to the step of placing said tube over a mandrel.

30. A stent comprising:
a tube having a pattern wherein said pattern has tri-fold symmetry forming a hexagonal grid pattern.

31. The stent of claim 30 where the wall of said tube is patterned for longitudinal flexibility where said pattern is comprised of a pattern of interconnected solid areas defining open spaces therebetween.

32. The stent as claimed in claim 31 where said pattern is a non-uniform pattern having first portions in which the open spaces defined by solid areas is sufficiently open to allow for passage of blood to branches and micro-arteries and having second portions where the open spaces defined by solid areas is sufficiently closed to allow formation of a thrombus.
